(19) United States
(12) Patent Application Publication
(10) Pub. No.: US 2021/0196446 A1
(43) Pub. Date: Jul. 1, 2021

(54) VASCULAR PROSTHESIS FOR USE IN THE TREATMENT OF ARTERIAL DISEASES

(71) Applicant: Mariano VICCHIO, SCANDIANO (RE) (IT)

(72) Inventor: Mariano VICCHIO, SCANDIANO (RE) (IT)

(21) Appl. No.: 16/756,504

(22) PCT Filed: Oct. 23, 2018
(86) PCT No.: PCT/IB2018/058239

§ 371 Date: Apr. 16, 2020

(30) Foreign Application Priority Data
Nov. 16, 2017 (IT) 102017000131109

(51) Int. Cl. A61F 2/06

(52) U.S. Cl. CPC A61F 2/064; A61F 2002/061; A61F 2002/065; A61F 2220/0016; A61F 2250/0098; A61F 2230/0067; A61F 2250/0039; A61F 2250/0063; A61F 2220/0075

(57) ABSTRACT

Disclosed is a vascular prosthesis made of biocompatible material including at least one first portion that is tubular and resilient at least in longitudinal sense. At least the tubular and resilient first portion has at least one reinforcement frame of biocompatible material shaped like a cuff-like covering, the reinforcement frame being adapted to allow the execution of at least one suture line with separated stitches for the stable and durable anchoring of the prosthesis to the vascular core of interest, the reinforcement frame defining, with the prosthesis portion from which it departs, an interspace, the latter being delimited by the external surface of the prosthesis portion enclosed by the reinforcement frame and by the internal surface of the latter.

### FIELD OF THE INVENTION

The present invention relates to the medical field and in particular to the cardiac surgery field. More in detail, the present invention relates to a peculiar vascular prosthesis adapted to the replacement of aortic segments, or arteries in general, involved in pathological processes. Said prosthesis allows consistently reducing the risk of intra-operative bleeding along the suture line between the artery and the prosthesis and minimizing the probability of detachment of long-term prosthesis.

### PRIOR ART

The use of vascular prostheses is part of a routine use method in the surgical correction of arterial diseases. The most frequent ones are aneurysms, stenosis and dissections. Tears due to trauma or fractures are instead more infrequent.

Prostheses used for the treatment of the aforesaid diseases are used specifically to replace the damaged artery segment. The prosthesis is positioned, by suture, between the two arterial stumps, after the removal of the damaged section. The current prostheses substantially exhibit two types of complications; one that occurs during the operation or in the immediate post-operative period, and the second one that occurs after a period of time, months or years. The complication that occurs immediately after implant of the prosthesis is represented by intra-operative or post-operative bleeding. In large-caliber arteries such as the aorta, especially in the case of dissection, a disease that alters the normal structure of the arterial wall, the implant of a vascular prosthesis involves the onset of a high risk of bleeding along the suture line.

Over the past several decades, various surgical techniques have attempted to reinforce the suture line between the aorta and the vascular prosthesis.

Attempts have been made to reinforce the suture line with pericardial strips, while other authors have described the use of Teflon or Dacron strips positioned inside and outside the aorta. Other authors used biological glues to recompose the wall of the aorta before implanting the prosthesis.

These techniques take time to prepare the aortic stump. In addition, the internal layer of Dacron increases the risk of thrombosis. However, despite these techniques, the problem of bleeding occurs in a still high percentage of cases.

In addition to the problem of bleeding, the prostheses currently used and the related implant techniques exhibit an additional long-term post-operative complication, represented by the detachment of the prosthesis with formation of a pseudo-aneurysm.

The formation of a pseudo-aneurysm at an implanted prosthesis is mainly due to the breakage of the suture thread that joins the prosthesis to the arterial stumps to which it is sutured.

The breaking of the suture thread causes the detachment of the prosthesis and the progressive dilatation of the scar tissues that have formed over time between the prosthesis and the arterial stumps. The formation of this dilatation requires a new operation when diagnosed in due time, otherwise spontaneous rupture occurs and therefore the patient's death.

The detachment of the prosthesis, with the formation of a pseudo-aneurysm, is an event that occurs in a high percentage of cases, since the prostheses currently used provide that their implant is carried out by means of a continuous suture, i.e. carried out with a single thread of suture. Therefore, all the parietal tension exerted by the arterial pressure present in the aorta, and therefore also in the prosthesis, must be contained by a single suture thread.

More in detail, at present, with the vascular prostheses on the market, the connection between the latter and the damaged artery can be carried out by means of a single suture line, with artery and prosthesis that are positioned in continuity, one following the other.

In the event of bleeding along this suture line, there is no possibility of positioning a second suture line. Usually, an attempt is made to place other points detached for hemostatic purposes along the single suture line, often causing an asymmetry of the suture, which is deformed in its structure with worsening of the bleeding.

For this reason, some surgical schools have introduced the use of Teflon, Dacron or pericardium strips in order to have a support on which to pass reinforcement stitches in case of bleeding. However, these techniques did not result in satisfactory results in the reduction of post-operative bleeding. It also takes a long time to prepare the arterial core with Teflon, Dacron or pericardium strips.

In order to understand the important problem that the present invention aims to solve, it is of interest to note that, as mentioned above, current prostheses are sutured to the artery with a single suture line. It is a continuous suture, represented by a single thread of synthetic material. The breaking of the latter, which in a certain percentage of cases takes place after some time, causes the detachment of the prosthesis from the aorta, with the formation of a pseudo-aneurysm.

The diagnosis of pseudo-aneurysm involves the need for a new surgical procedure. In a certain percentage of cases, the pseudo-aneurysm undergoes spontaneous rupture with the patient's death.

The development of the first vascular prostheses began in the sixties, following the experimental studies conducted in the previous decade.

Examples of vascular prostheses that are used in the treatment of the aforesaid aortic diseases are described in several prior art documents: document U.S. Pat. No. 3,011,527 is among the first ones to describe a tubular prosthesis made of woven material, Dacron, with a specific organization, called woven, of the fibers that make up the fabric.

Document U.S. Pat. No. 3,316,557 describes a tubular prosthesis for replacing damaged arterial segments, made of fabric material internally coated with animal collagen in order to reduce the porosity of the fabric and hence the transudation of blood from the prosthesis.

US 2004/0193244 discloses a vascular prosthesis for the treatment of aortic aneurysms comprising a tubular portion of a biocompatible material having a proximal and a distal end. From the latter, at least one self-expanding portion branches off.

Document AU2010254599 B1 describes for example a hybrid prosthesis particularly useful for use thereof in the treatment of dissections of the aorta.

This prosthesis is hybrid in that it consists of a classic tubular portion, made of woven material, which is positioned at the ascending aorta, and an expandable endoprosthetic portion, positioned in the arch and in the descending aorta. The feature of this prosthesis lies in that the endoprosthetic portion provides a recess to allow perfusion of the arterial trunks that arise from the aortic arch.

Document EP2606853 likewise describes a hybrid prosthesis for use in the treatment of vascular diseases and in particular for the treatment of aortic aneurysms.

This prosthesis is also made up of a typical portion of fabric and an endoprosthetic portion, consisting of an expandable metal structure, called a stent, covered with fabric. The feature of this prosthesis is the presence of a branch in the expandable portion, also provided with an expandable stent, designed to be opened inside an arterial vessel that departs from the aorta, ensuring the perfusion thereof.

Document FR 2850008 also describes a vascular prosthesis particularly adapted for the treatment of vascular diseases and in particular a prosthesis adapted to adjust to differences in diameter between the two ends of a blood vessel to be replaced, typically between the two ends of a pathological aortic trait previously removed. It is basically a structure having a prosthetic tubular portion provided, at one end thereof, with a portion shaped as a planar collar or flange, adapted to adjust to the different diameter extension of the blood vessel core, typically aortic, which is variable from subject to subject.

Document GB 2427554 describes a peculiar device particularly adapted for the treatment of aneurysms of the aortic arch and of the descending thoracic aorta. Also this prosthesis has a ring along the body of the prosthesis. This is a planar element, like the prior art described above.

This prosthesis is used for a precise intervention called “elephant trunk intervention”. This surgical technique provides that, at first, the distal tubular portion of the prosthesis is left within the descending thoracic aorta. As the descending thoracic aorta is affected by the aneurysm, its caliber, in this section, will be significantly increased compared to the prosthesis. The suture of the distal aortic stump around the prosthesis is made, using the prosthesis described in GB 2427554, using the large diameter planar ring, which is shaped according to the diameter of the aneurysm.

The suture that can be made with this ring is a single suture, and this ring does not have the advantages of stabilization over time, and hemostasis, which instead the subject prosthesis ensures due to its essential features explained in the following description.

The second surgical time of the use of the prosthesis described in GB 2427554 is represented by the positioning of an endoprosthesis through the femoral artery. For this purpose, the prosthesis has a series of radio markers placed on the distal portion of said device, which are fluoroscopically visible, and adapted to identify the correct positioning of the endoprosthesis, which is positioned as a telescope in the distal tubular portion of the prosthesis.

US 2001/0049553 describes a vascular prosthesis designed for its implant at the aortic root. Said prosthesis, in order to avoid stretching of the coronary arteries, comprises a first tubular portion and a second tubular portion connected to the first one and coaxial thereto. Said second tubular portion mimics the normal function and anatomy of the valsalva sinuses, from which the coronaries originate.

This second portion, having a larger diameter than the first portion, favors the suture of the coronary arteries, avoiding the stretching thereof.

Although the sophistication and effectiveness of the prosthetic devices described in the aforementioned prior art documents is undoubted, in their adaptation to the space regions corresponding to the pathological aortic sections, the bleeding problem that can be found at the suture line between the ends of the prosthesis and the vascular cores is still unresolved. As mentioned above, this bleeding is substantially derivable from the fact that the prosthesis is fixed by suture and, specifically, by a single continuous suture which, to date, represents the classical suture modality between the prostheses currently on the market and the aortic abutment.

This suture is typically performed using a single suture thread which is passed between the prosthesis and the aortic stump numerous times, until the whole circumference of the prosthesis is anchored to the entire circumference of the aortic stump. Often, when making the implant, an additional portion is used, represented by a strip of Teflon used to reinforce the suture line and to prevent bleeding. Some authors also place one internally in the aorta. These Teflon strips also allow placing further sutures for hemostasis in the event of bleeding. Nevertheless the problem of bleeding along the suture line is not yet completely resolved.

In this regard, the object of the present patent application for industrial invention is to propose a particular prosthesis, to be implanted in regions corresponding to pathological vascular traits removed, typically aortic, which, compared to conventional prostheses, has structural features such as to avoid all the above criticalities associated with bleeding along the suture line between the prosthesis and the vascular core.

### DESCRIPTION OF THE INVENTION

The present description relates to a peculiar type of vascular prosthesis, for use in the treatment of vascular diseases, and typically for use in the treatment of aortic aneurysm and aortic dissection.

More in detail, the present description relates to a vascular prosthesis, for use in the treatment of aortic aneurysm, which has implemented features, with respect to conventional prostheses, aimed to reduce or even prevent the onset of the risk of intra-operative bleeding along the suture line between the prosthesis and the aortic stump involved, and to reduce the risk of detachment of the prosthesis and the formation of pseudo-aneurysms in the long term. Even more in detail, the subject prosthesis includes structural features that allow consistently innovating the current anchoring techniques of the prosthesis to the aortic stump, allowing the anchoring to be made definitely stable and durable, thus avoiding any possible critical issues arising from the implant including, first of all, the formation of pseudo aneurysms, often resulting from the characteristic and usual anchors for the implant of conventional prostheses.

Specifically, and similarly to traditional prostheses, the object of the present invention comprises at least one tubular portion resilient at least in the longitudinal direction. Said prosthesis is characterized in that it has a cuff-like covering portion, hereinafter referred to as a prosthetic reinforcement “frame”, on at least one of the ends of said resilient tubular portion, which defines an essential interspace between the body of the prosthesis and the frame that departs therefrom. It is therefore of interest to point out that with respect to the aforementioned prior art document FR2850008, which describes a vascular prosthesis provided with a collar-like component, the subject prosthesis, with its frame, has a substantial difference: the collar described in said prior art document is in fact a planar structure which extends perpendicularly to the longitudinal axis of the tubular body of the prosthesis, thus preventing the formation of an interspace between said collar and the prosthesis. Likewise, also document GB 2427554 describes a prosthesis provided with a planar ring which, in addition to not allowing the definition of the essential interspace provided by the frame of the subject prosthesis, is not positioned at the end of the prosthesis, thus performing a completely different function from that performed by the frame of the prosthesis according to the present invention.

Substantially, said prosthetic reinforcement frame allows providing a valid support for suturing the prosthesis to the stump, not exclusively with a conventional and single suture line but with a special suture with detached stitches which can be made with numerous double needle suture threads. This possibility allows obtaining a fixation of the prosthesis which is decidedly more stable in the long term and preventing the risk of consistent intra-operative bleeding along the suture.

More specifically, the prosthesis of the present invention comprises at least a first portion, represented by a traditional resilient tubular portion, by way of non-limiting example a resilient and corrugated tubular portion made of a biocompatible material. Alternatively, smooth prostheses such as those used for small-caliber, peripheral (femoral, etc.) arteries may also be representative. In some embodiments said prosthesis includes structural variants which make it particularly suitable for its the implant thereof at specific portions of the aorta. For example, some embodiments according to the present invention provide that said prosthesis includes some branches which make it adapted for the implant thereof at the aortic arch, or which has a segment shaped as a bifurcation to be used in the abdominal aorta site, at the bifurcation thereof at the aortic carrefour, or comprising a second portion, connected to the first without interruption, represented by an endoprosthesis, i.e. a portion in the shape of a self-expanding stent for the implant at the descending thoracic aorta.

Independently of the additional features of said embodiments, the subject prosthesis is characterized first of all by the presence, on at least one end of said resilient tubular portion, of at least a portion shaped like a frame which makes the anchoring of the prosthesis to the aortic stump effectively and permanently stable, also reducing the risk of intra-operative bleeding along the suture.

More in detail, as mentioned above, said frame appears as an extension in the form of a cuff-like covering of the tubular portion of the prosthesis.

This frame, connected to said tubular portion without interruption, therefore appears as a cylindrical or frustoconical structure in which two bases can be identified: a first base and a second base, where first base means that in continuity with the tubular portion of the prosthesis and closer to the end of the aortic abutment on which said prosthesis is implanted at said frame. If said profile of the frame is frustoconical, said first base has a diameter smaller than that of the second base, and coincides with that of the tubular portion, from which said frame departs. The second base instead has a larger diameter than that of the first base and that of the tubular portion, and its margin is not in continuity with the prosthesis, but is free. Said frustoconical profile of the frame thus defines an interspace delimited by the outer surface of the tubular portion surrounded by the frame and the inner wall of the latter. If said profile of the frame is cylindrical, the first base and the second base will have a diameter of equal extension which, in order to ensure the definition of the above interspace, will be slightly larger than that of the tubular portion from which the first base of said frame departs. Typically, but not necessarily or limitedly, the diameter of the first base and second base is at least 0.3 mm greater than that of the tubular portion.

Advantageously, the subject prosthesis, due to said frame, is suturable to the artery with numerous stitches like an upturned “U”. It is therefore a suture with detached stitches, where the parietal tension, generated by arterial pressure, is distributed over multiple suture threads. In this way, the possibility of breaking a suture thread is reduced.

Advantageously, even if a thread should accidentally break, being the suture formed by other stitches, the latter will hold the prosthesis in place, reducing the risk of a reoperation.

Advantageously, the profile and the extension of the frame characterizing said prosthesis allow the possibility of a second suture line with greater stabilization of the prosthesis over time and reduction of intra-operative bleeding.

Advantageously, the presence of the aforementioned interspace existing between the inner surface of the frame and the outer surface of the prosthetic tubular portion wrapped by the said frame, offers the surgeon the possibility of suturing the prosthesis to the stump in various ways, allowing, depending on the case, to evaluate and choose to suture the prosthesis to the stump in the most suitable way to ensure greater stability of the implant.

This interspace allows the positioning of additional sutures for hemostatic purposes, in the event that after implantation of the prosthesis, bleeding occurs along the suture line between the aortic stump and the prosthesis.

### BRIEF DESCRIPTION OF THE DRAWINGS

The invention which is the object of the present patent application for an industrial invention will hereinafter be described in detail in preferred embodiments thereof and with reference to the accompanying figures, in which:

FIG. 1 shows an explanatory view of the implant of the first tubular and resilient portion 1′ of the vascular prosthesis 1 at the end of a typically aortic vascular core portion 100. More in detail, the subject figure shows the presence of the reinforcement prosthetic frame 2, which characterizes the prosthesis 1, and how the stitched suture is made with numerous double needle suture threads 3. Each suture thread forms a sort of inverted “U”. Said figure shows that numerous inverted “U” stitches are positioned, thereby forming a first suture line.

FIG. 2 shows an evolution of FIG. 1 in which the prosthesis 1 was pushed along the detached stitches which are held in tension by the surgeons, resting within the aortic core. In the subject figure, the reinforcement frame 2 has a cylindrical profile.

FIG. 3 shows the detached stitches knotted one by one and the cut suture threads.

FIG. 4 shows how a further suture thread is used to make a further continuous suture along the upper margin of the aortic stump 100 and of the prosthetic reinforcement frame 2. The subject figure aims to highlight how the reinforcement frame 2 allows the execution of two different suture lines, a lower and an upper one in which the lower line 5 is preferably to be made with detached suture stitches. The upper suture line 6 shown in the subject figure is represented by a continuous suture, by way of example, but it may also be represented by an additional suture line with detached stitches.

FIG. 5 shows an evolution of the preceding FIG. 4. More in detail, the subject figure shows the first tubular and resilient portion 1′ of prosthesis 1 lying in the aortic core, with the two completed suture lines. The lower suture line 5 with detached stitches and the upper line 6 consists of a continuous suture.

FIG. 6 shows a detailed view of the implantation of the subject prosthesis 1 to the aortic stump 100 and in particular how the prosthetic reinforcement frame 2 characterizing said vascular prosthesis 1 is spatially configured. The subject figure aims in particular to highlight the formation of the interspace 7 which is created between the outer surface of the first tubular and resilient portion 1′ of the prosthesis 1 wrapped by the prosthetic reinforcement frame 2 and the inner wall of the latter. Said interspace 7 also facilitates the suture by the operating surgeons.

FIG. 7 shows a detailed view of the prosthesis 1 when implanted and in particular of the first lower suture line with detached stitches and the second upper suture line represented by a typical continuous suture. The subject figure also shows that said first lower suture line with detached stitches involves only the reinforcement frame 2 and corresponding points on the aortic core, leaving the wall of the first tubular portion of the prosthesis 1 free.

FIG. 8 is similar to the preceding FIG. 7 but, unlike this, shows the case in which the first lower suture line with detached points involves the aortic core wall, the reinforcement frame 2 and corresponding stitches present on the wall of the first tubular portion 1′ of the prosthesis surrounded by said reinforcement frame 2. The aim is to achieve a more robust suture. In this case, it is necessary that the suture be made at the bottom and in particular where the reinforcement frame 2 departs from the tubular portion. In this way, adequate mobility of the tubular portion with respect to the frame during the steps in which the surgeon should work in that space could be ensured.

FIG. 9 shows a detailed and explanatory view of a particular embodiment according to the present invention. More in detail, FIG. 9(a) shows the case in which, from the area in which the reinforcement frame 2 extends from the wall of the first tubular and resilient portion of the prosthesis, a further portion branches off, and in particular a portion extension 8 in continuity with said prosthetic frame. On said lower extension 8 it is possible to make the suture, and in particular the first suture line with detached stitches. The reinforcement frame is configured to overhang the edge of the aortic stump. In this case, the reinforcement frame 2 has longitudinal cuts 9 (FIG. 9(b)) through which it can be folded, thereby covering the outer wall of the aortic stump.

FIG. 10 shows the evolution of the preceding FIG. 9. The subject figure shows in particular the reinforcement frame 2 folded on the outer wall of the aortic core and sutured thereto with a continuous suture line. The figure also shows the detached stitch suture involving the lower extension 8 of the frame 2 and the aortic core 100.

FIG. 11 shows an explanatory representation of a further embodiment of the vascular prosthesis 1 according to the present invention. More in detail, the subject figure shows the case in which said prosthesis in addition to the lower extension 8 is provided with a reinforcement frame 2 whose length is such as to allow not only said frame 2 to be folded by wrapping the outer surface of the aortic stump 100, but also to extend up to cover the entire area of the aortic stump which is in contact with said lower extension 8. In this way, the first lower suture line with detached stitches can be very robust, involving: the lower extension 8, the aortic core 100 and the reinforcement frame 2. The purpose of this embodiment is to provide a particularly robust suture by imitating the structure that some surgeons currently perform using Teflon or Dacron strips.

FIG. 12 compares two embodiments of the prosthesis 1 according to the present invention. More in detail, FIG. 12(a) shows said prosthesis 1 comprising the first tubular and resilient portion 1′ provided, on at least one of the ends thereof, with the prosthetic reinforcement frame 2 (which in the subject figure has a frustoconical profile), adapted to accommodate the detached suture stitches in the shape of inverted “U”, while FIG. 12(b) shows the case in which these stitches are already pre-assembled, meaning that the suture threads are already assembled to said prosthesis. This embodiment is particularly advantageous in that it allows the surgeon to perform, during the implantation of the prosthesis, only one movement consisting in passing the needles in the artery alone rather than in the prosthesis wall and then in the artery, thus reducing the time normally required to make the implant.

FIG. 13 shows a perspective view of a particular embodiment of the subject prosthesis. More in detail, the subject figure shows the case in which the prosthesis 1 has a plurality of reinforcement frames 2. More in detail, the subject figure shows that in addition to a first reinforcement frame present on one of the two ends of the first tubular and resilient portion 1′ of the prosthesis 1, there are further reinforcement frames 2 which extend one above the other and which are interspersed with portions of said first tubular portion 1′. The aim of this embodiment is to allow the cardiac surgeon, before implanting the prosthesis 1, to evaluate exactly the ideal length of the prosthesis portion to be implanted, thus cutting said prosthesis to a predetermined height. The subject figure further shows that also at the other end of said first tubular portion 1′ of the prosthesis 1, there is a reinforcement frame 2, adapted to perform exactly the same function performed by the frame present at the other end, that is, ensuring a durable and stable anchoring of the prosthesis at the end of the other stump portion of the pathological artery.

As is intuitive and observable in the subject figure, the reinforcement frames which branch off from two opposite ends of the prosthesis portion are specular.

FIG. 14 shows a representation of a further embodiment according to the present invention. More in detail, the subject figure shows the case in which the prosthesis 1 comprises, on the body of its first tubular and resilient portion 1′, having at least a reinforcement frame 2 at one of the ends thereof, ramifications 10 in the form of side branches. This embodiment shows the case in which the prosthesis 1 is adapted to replace the removed pathological aorta and allow the perfusion of one or more arterial trunks, which depart therefrom, due to said ramifications 10.

FIG. 15 shows a variant of the prosthesis shown in the preceding FIG. 15. More in detail, the subject figure shows the case in which the prosthesis comprises a first tubular and resilient portion 1′ having the ramifications 10 and a plurality of reinforcement frames 2. As shown in the figure, the reinforcement frames present at the proximal end of the portion 1′ and those present at the distal end of the portion 1′ are specular, where the proximal end is the one closest to the heart and the distal end is that farthest from the heart. The embodiment shown in the subject figure allows the surgeon to cut the prosthesis to the required length so that predefined reinforcement frames can be adapted to the aortic stumps whose distance varies from patient to patient.

FIG. 16 shows a further variant of prosthesis 1. More in detail, the subject figure shows the case in which said prosthesis comprises a first tubular and resilient portion 1′ and a second portion 1″ represented by an endoprosthesis or an expandable portion with a metal stump capable of opening in the aorta. In this representation, a plurality of reinforcement frames 2 are visible on the first tubular and resilient portion 1′ of the prosthesis 1 which allow the surgeon to choose at which point it is convenient to cut said first tubular portion of the prosthesis in order to ensure the most correct adaptation thereof, a function of the distance between the two aortic stumps between which said prosthesis 1 must be implanted.

FIG. 17 is similar to the preceding FIG. 16. With respect to the latter, the subject figure shows the presence of a reinforcement frame 2 at the transition point between 1′ and 1″ of the prosthesis 1 at the other end of the first prosthetic tubular portion 1′. The aim is always to ensure greater anchoring stability of the prosthesis to the distal aortic stump, and not only to the proximal one.

FIG. 18 shows the case in which the prosthesis 1 according to the present invention comprises a first tubular and resilient portion 1′ with three reinforcement frames 2 with a frustoconical profile which branch off from one end of said first portion 1′ of the ramifications 10 for the perfusion of the arterial trunks, and a second portion 1″ in the form of a self-expanding stent. It is also possible to see a reinforcement frame 2, mirroring the aforesaid other three reinforcement frames, at the transition point between said first portion 1′ and the second portion 1′ of prosthesis 1.

FIG. 19 is similar to the preceding FIG. 18. With respect to the latter, the subject figure shows the case in which the subject vascular prosthesis comprises: a first tubular and resilient portion 1′ with a plurality of reinforcement frames 2, the ramifications 10 for perfusing the arterial trunks and a second portion 1″ represented by a self-expanding stent, in which at the transition point between the first portion 1′, and second portion 1″ of prosthesis 1 a ring 11 can be found adapted to suture the prosthesis to the aorta in its descending portion which can be very dilated. Said ring is already found in commercially available vascular prostheses.

FIG. 20 shows the case in which the first portion' of prosthesis 1 has, in its distal segment, also some position indicators. More in detail, FIG. 20(a) shows a prosthesis 1 comprising a first tubular and resilient portion 1′ having a plurality of reinforcement frames 2, the ramifications 10 for perfusing the arterial trunks and position indicators, or radio markers 12. FIG. 20(b) is similar to FIG. 20(a) but also shows the presence of a branch 13 for intra-operative perfusion of the prosthesis.

FIG. 21 shows a peculiar embodiment of the prosthesis according to the present invention in which reinforcement frames 2, suitably sized, are also found on the ramifications 10 for perfusing the arterial trunks.

FIG. 22 shows the case in which said prosthesis comprises a first tubular and resilient portion 1′ having at least a reinforcement frame 2, specifically four reinforcement frames 2, and radio markers 12.

FIG. 23 is similar to the preceding FIG. 22. With respect to the latter, the subject figure shows the case in which at the transition point between the first tubular portion 1′ of the prosthesis and the section comprising radio markers 12, a reinforcement frame 2 with a specular orientation to those of the reinforcement frames 2 which branch off sequentially from one of the ends of portion 1′ of the prosthesis 1 can be seen.

FIG. 24 shows the case in which the first portion 1′ of the prosthesis 1 is represented by a tubular and resilient portion with ramifications 10 and a branch 13 for perfusion of the prosthesis and position indicators 12 and reinforcement frames 2.

FIG. 25 shows an embodiment of the prosthesis 1 according to the present invention in which said prosthesis comprises a first tubular and resilient portion 1′ with at least one reinforcement frame 2, specifically three reinforcement frames 2, the ramifications 10 and the perfusion branch 13; and a second portion 1″ represented by a self-expanding stent. A reinforcement frame 2 is present at the point where said prosthesis is sutured to the distal core of the descending thoracic aorta.

FIG. 26 shows a further peculiar embodiment of the vascular prosthesis according to the present invention, wherein said prosthesis comprises a first tubular portion 1′ having at least one reinforcement frame 2, typically a reinforcement frame 2 at one end, and an integrated prosthetic valve at the other end.

FIG. 27 shows a particular embodiment according to the present invention in which the prosthesis 1 comprises a first tubular and resilient portion 1′ having a portion shaped as a bifurcation 14. Said prosthesis, in this embodiment, is adapted to the implant thereof in the seat of the abdominal aorta, at the aortic carrefour thereof. The reinforcement frames 2, appropriately sized, may also be present on said bifurcation.

FIG. 28 shows a particular embodiment according to the present invention in which the prosthesis 1 comprises on the body of the first portion thereof an ampoule dilatation adapted to mimic the shape of the aortic bulb to facilitate the suture of the coronaries on such a dilatation of the prosthesis, avoiding the stretching of the coronary arteries. At the other end of the prosthesis, at least one reinforcement frame 2 is present distal to the aortic bulb.

FIG. 29 is similar to the preceding FIG. 28, and shows the case in which the prosthesis 1 as described in the preceding figure is further provided, at the proximal end thereof, with an integrated prosthetic valve.

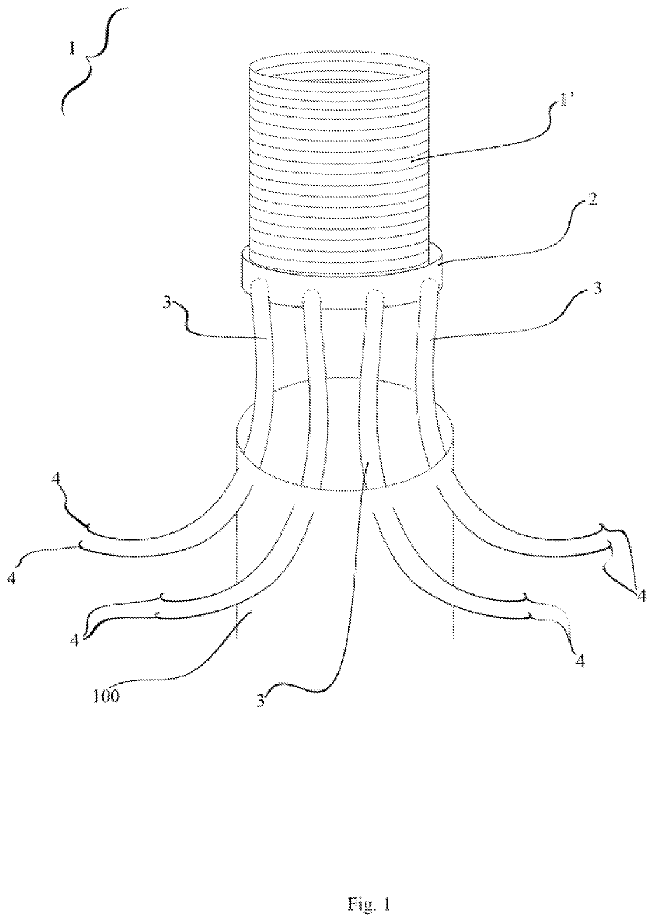

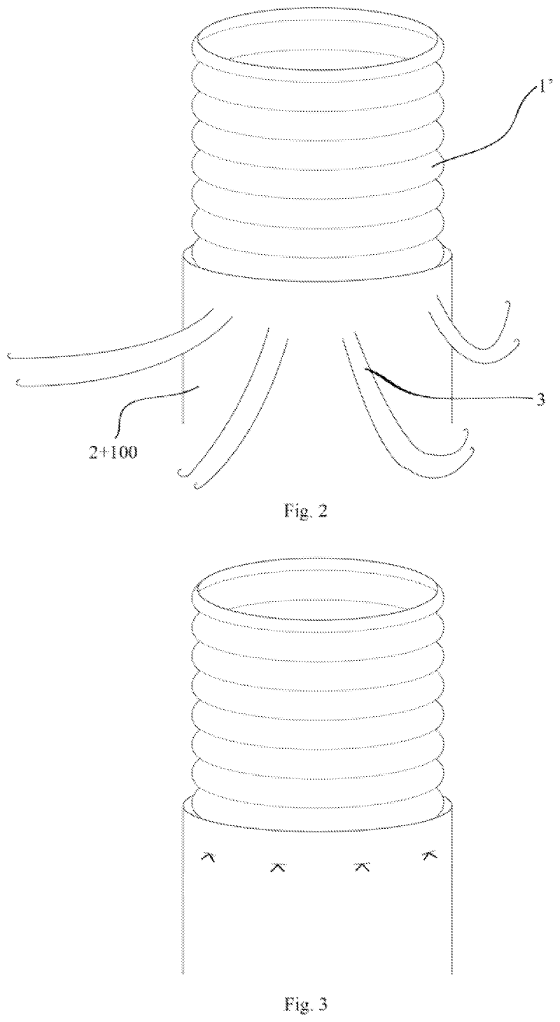

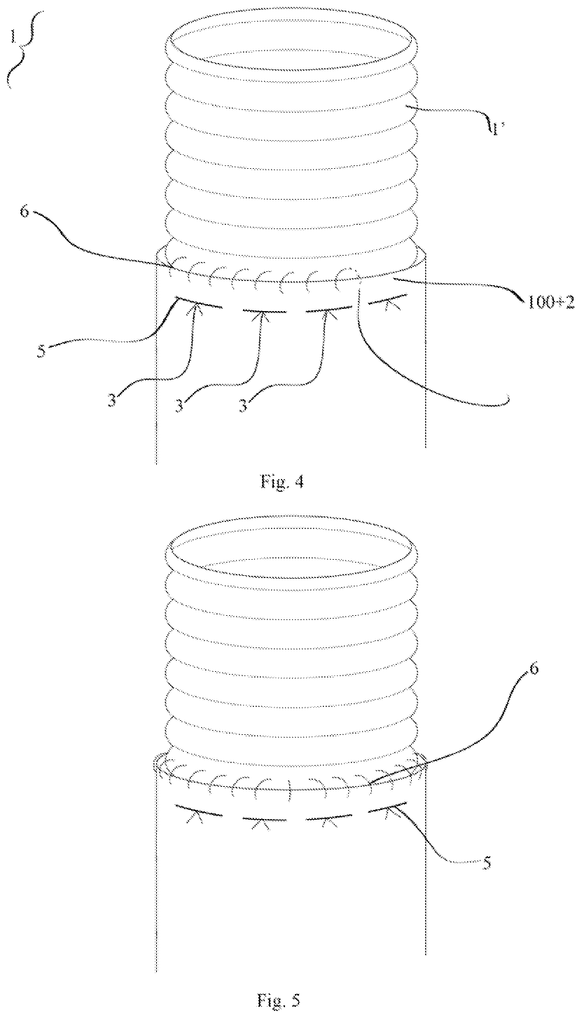

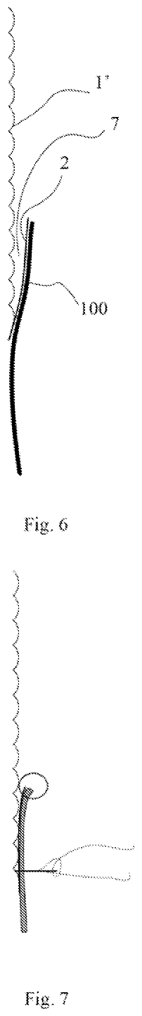

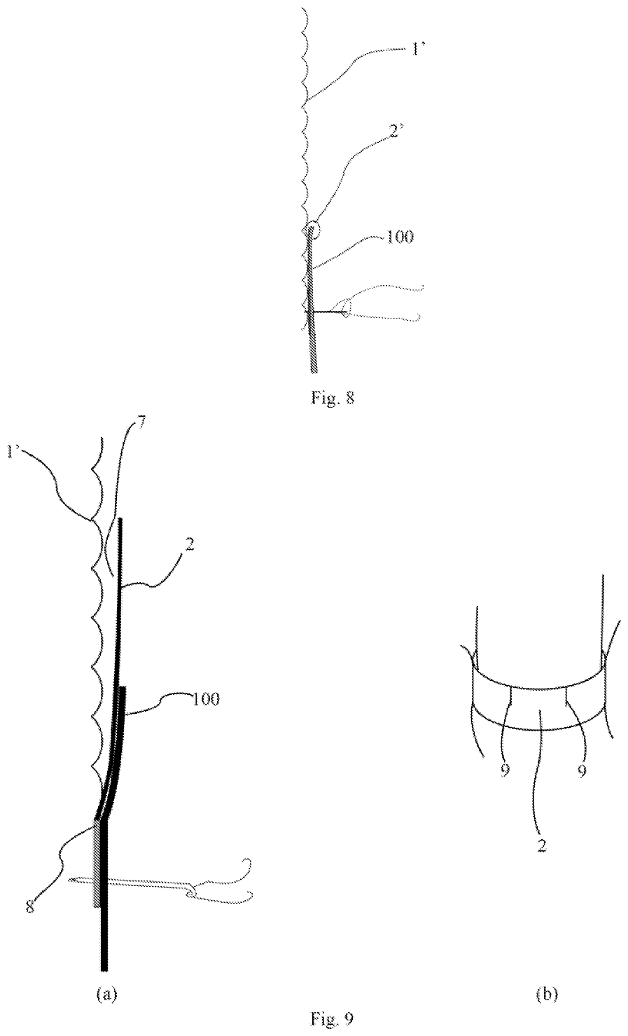

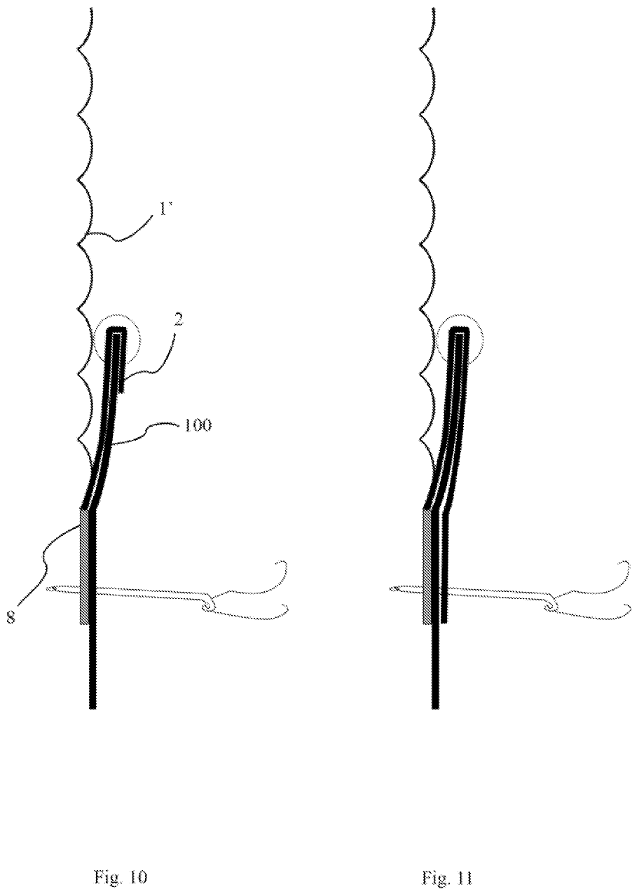

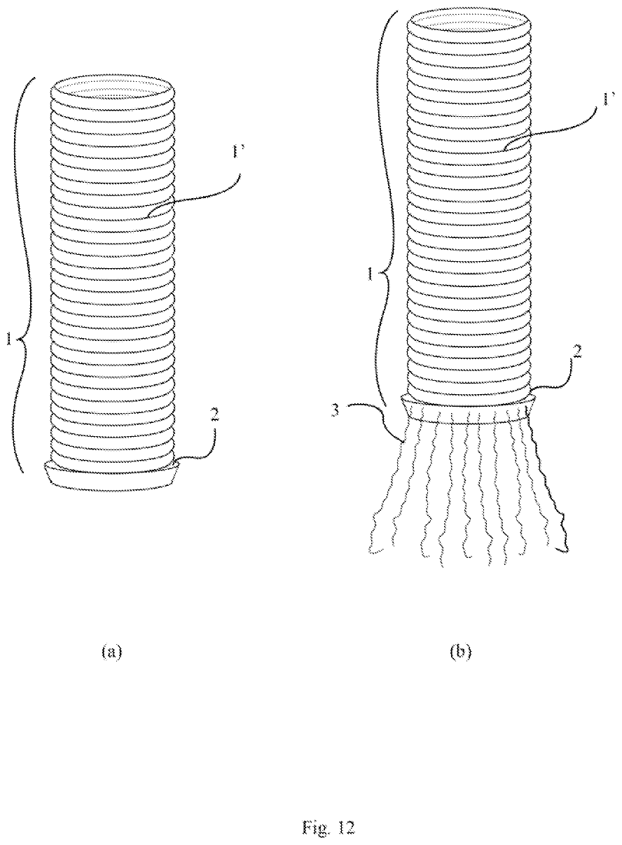

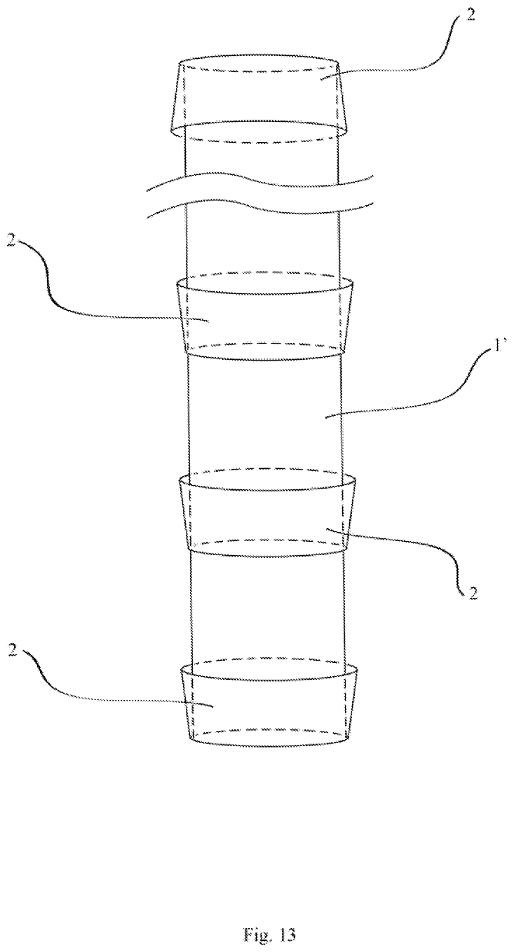

### DESCRIPTION OF THE PREFERRED EMBODIMENTS

In all of its embodiments, corresponding to the above figures, the vascular prosthesis 1, object of the present patent application for industrial invention, is characterized substantially in that it comprises at least one reinforcement frame 2 shaped like a cuff-like covering adapted to make the anchoring of the prosthesis itself easier, more stable and longer lasting at the implant site where said prosthesis replaces a pathological artery section. All this avoiding the onset of all the problems described above. First of all, the formation of pseudo-aneurysms involving the aorta.

The subject prosthesis 1 comprises at least one tubular and resilient portion 1′ on which at least one reinforcement frame 2 can be found. Specifically, a reinforcement frame can be found on at least one of the ends of said prosthesis portion 1′.

Further embodiments provide that said portion 1′ of prosthesis 1 comprises ramifications 10 which mimic the normal function of the arterial branches and/or that is has radio markers 12 or that it has a bifurcation structure 14 or that, in addition to said first tubular and resilient portion 1′, there is also a second portion 1″ of prosthesis 1, in the form of a self-expanding stent.

Depending on the peculiar structural variations found in the subject prosthesis 1, corresponding to its various embodiments, said prosthesis 1 is particularly suitable for use in the treatment of aortic diseases, in particular for use in treating the dissection or the aneurysm, located at the level of the aortic root; of the ascending thoracic aorta; of the aortic arch; of the descending thoracic aorta; of the abdominal aorta and for the extensions of the pathology involving more than one of the aorta portions mentioned above.

In all of its embodiments, said prosthesis is characterized in that it comprises at least one reinforcement frame 2, typically found on at least one end of at least one of its said first portion 1′. More in detail, the reinforcement frame 2 appears as a structure which branches off, without interruption, from the portion(s) on which it is found. It is a covering-like structure, like a cuff, with a cylindrical or frustoconical profile. In the latter case, said profile defines a first base whose diameter coincides with that of the prosthesis portion with a tubular profile from which it departs, and a second base, with a diameter greater than that of the first base so that said covering defines an interspace 7 delimited by the outer surface of the portion of prosthesis 1 from which said frame 2 departs and the inner surface of the latter. If the reinforcement frame 2 has a cylindrical profile, the first base and second base of the frame will have the same diameter, which will be slightly larger, in order to ensure the definition of the interspace 7, with respect to that of the tubular portion from which said first base departs.

In all of its embodiments, said reinforcement frame 2 allows implanting the prosthesis to the aortic stumps defining the region of space previously occupied by the pathological artery tract, in a significantly stable manner, allowing the surgeon to make at least a first suture line 5 of the type with detached stitches and optionally a second suture line 6, also with detached stitches or a traditional and continuous suture.

In all of its embodiments, the vascular prosthesis 1 may comprise suture threads 3 already integrated with said reinforcement frame 2 so that each suture thread 3 defines an inverted

In its simplest embodiment, the vascular prosthesis 1 comprises a first tubular portion 1 resilient at least in the longitudinal direction, and is characterized in that it comprises at least a reinforcement frame 2 which can be found on at least one of the ends of said first portion 1′ of the prosthesis. If said prosthesis has a reinforcement frame 2 also at the opposite end, said frame is configured so as to be specular to the first one. Both reinforcement frames 2 present on both ends of the first prosthetic portion 1′ are such as to allow their stable adaptation, by means of sutures with detached stitches, at the ends of the aortic stumps which define the implantation site of the prosthesis 1.

The prosthesis 1 thus described is particularly suitable for its use in the treatment of the ascending aortic aneurysm.

Further embodiments of the prosthesis 1 provide that the latter comprises ramifications 10 which simulate the function of the arterial branches of the aortic arch, or one or more radio markers 12 adapted to guide the telescope positioning of an endoprosthesis within the distal portion of the subject prosthesis, or comprising an additional portion, i.e. a second portion 1′ in the form of a self-expanding stent.

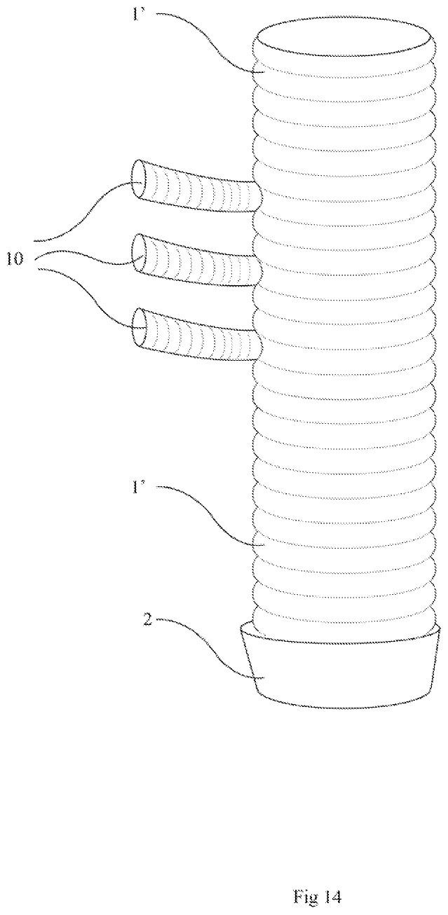

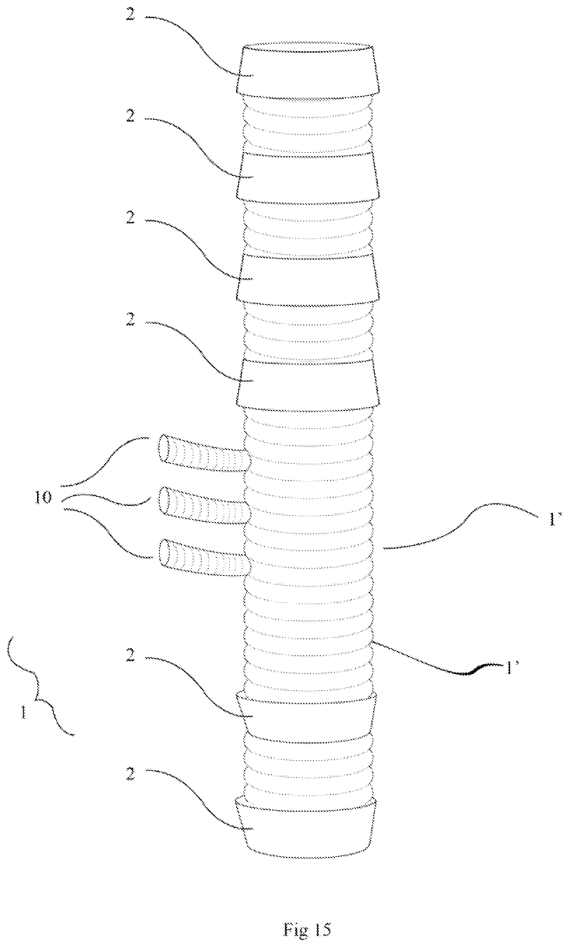

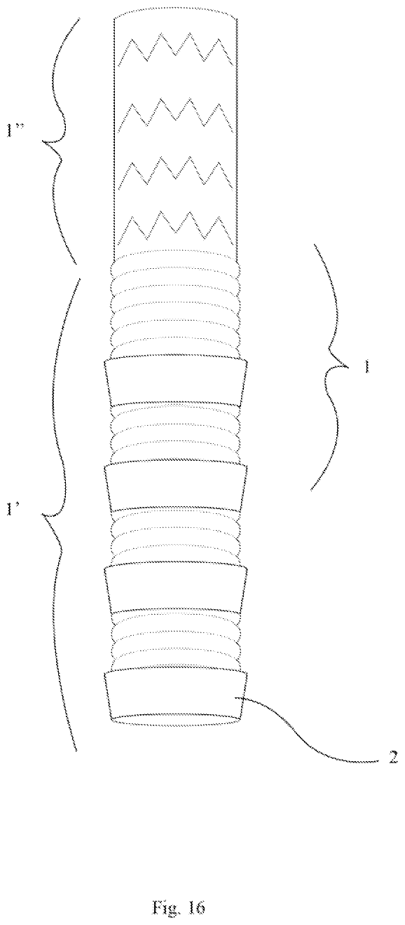

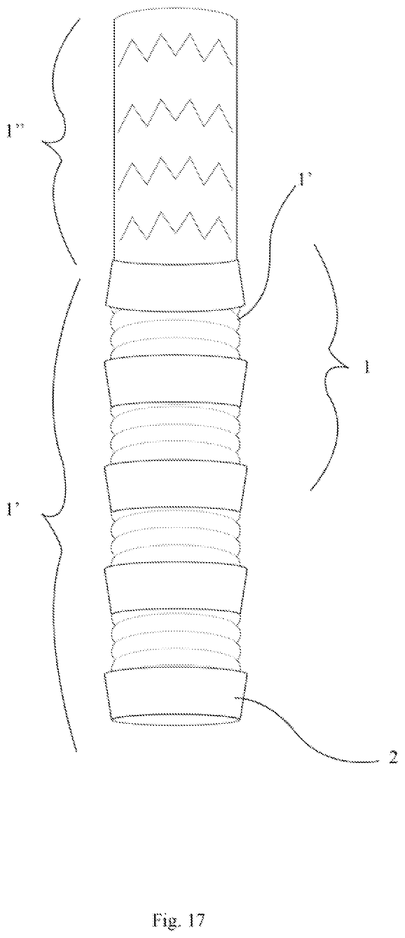

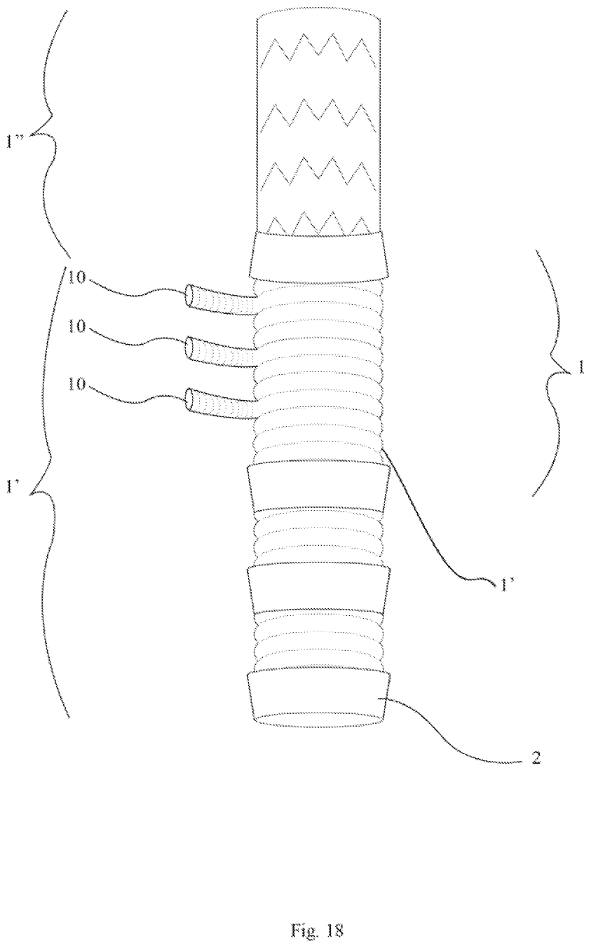

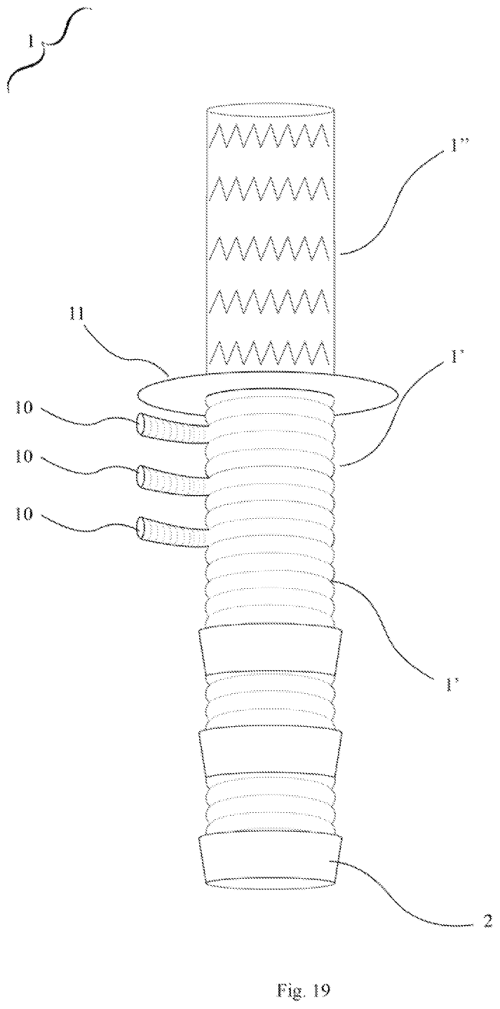

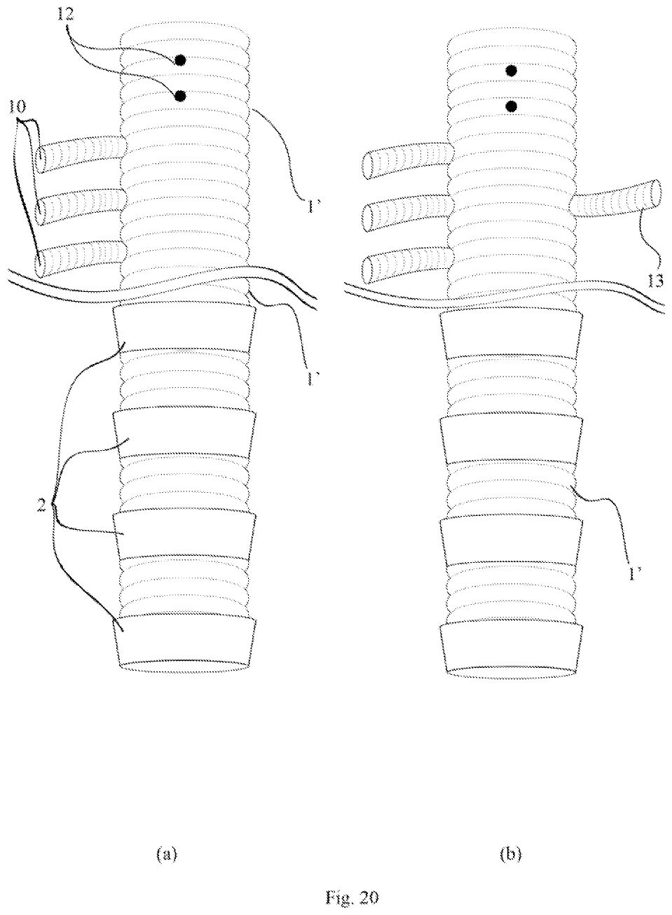

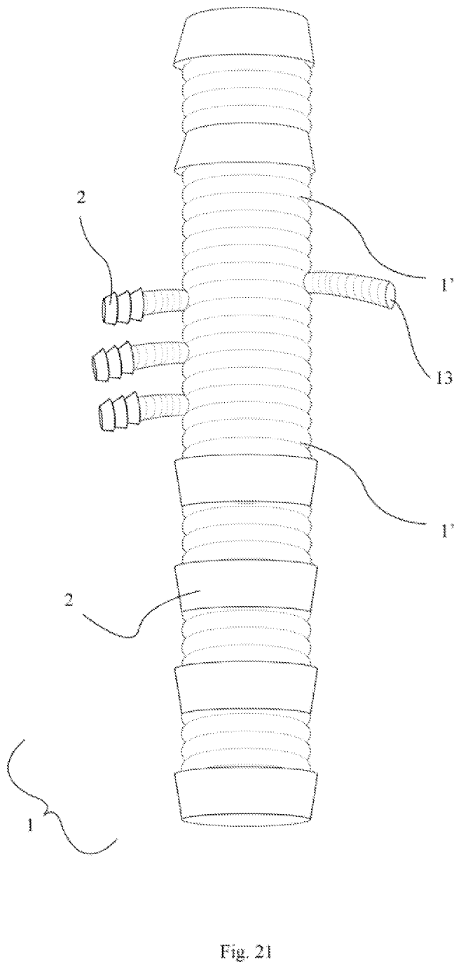

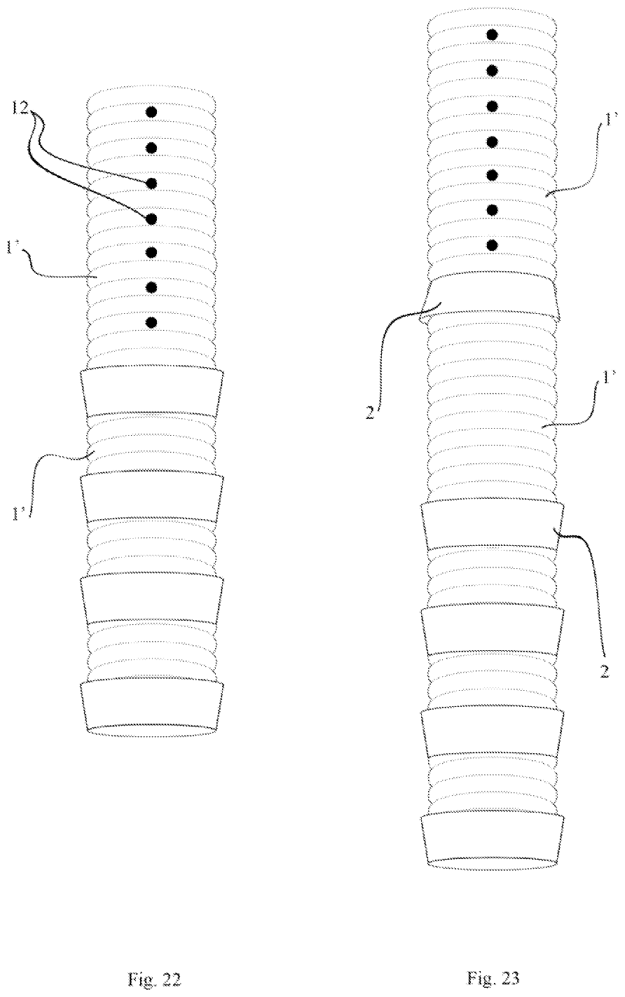

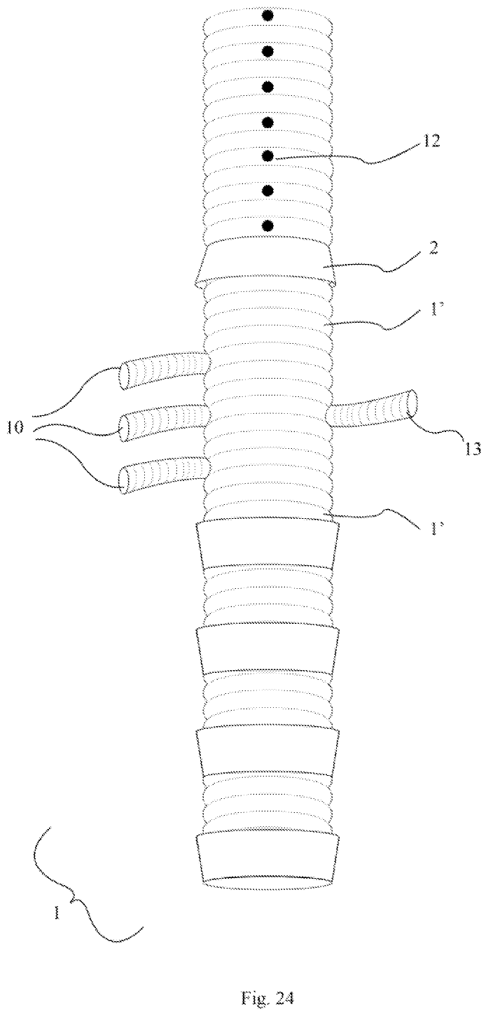

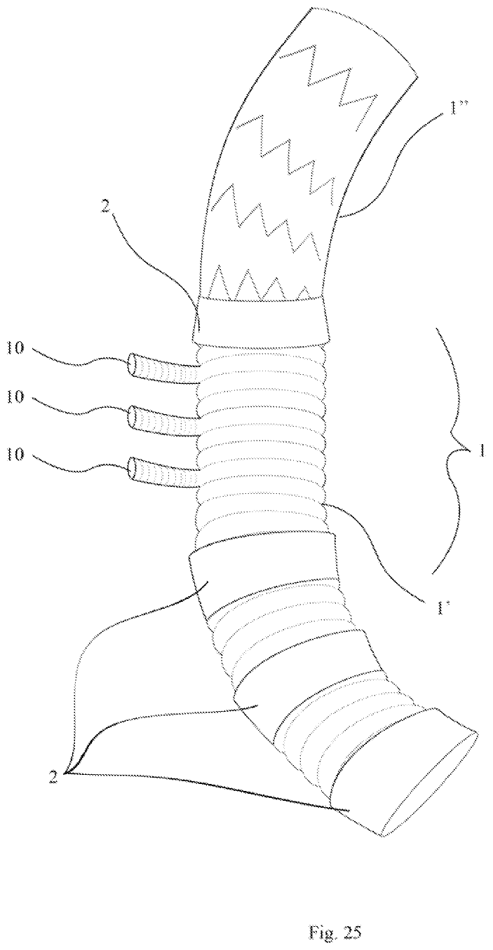

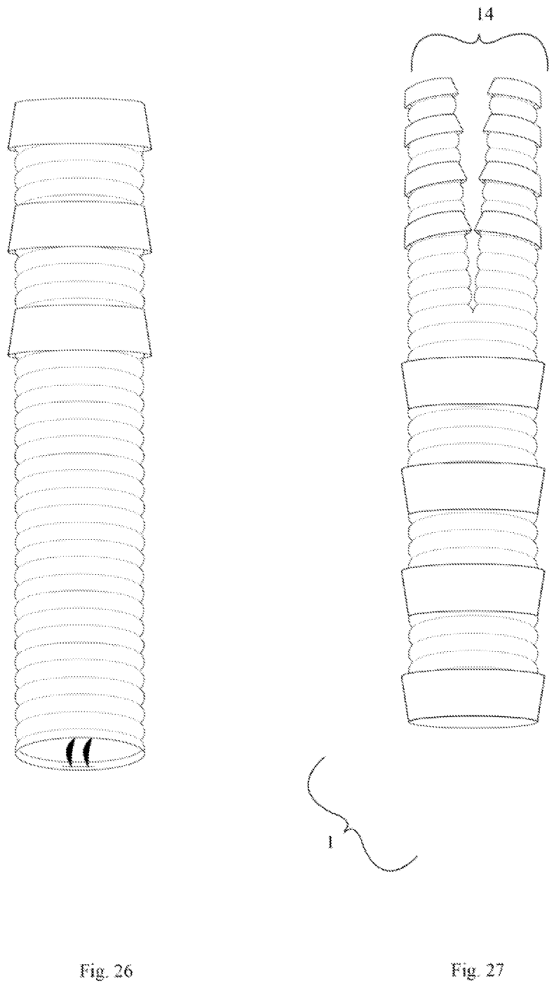

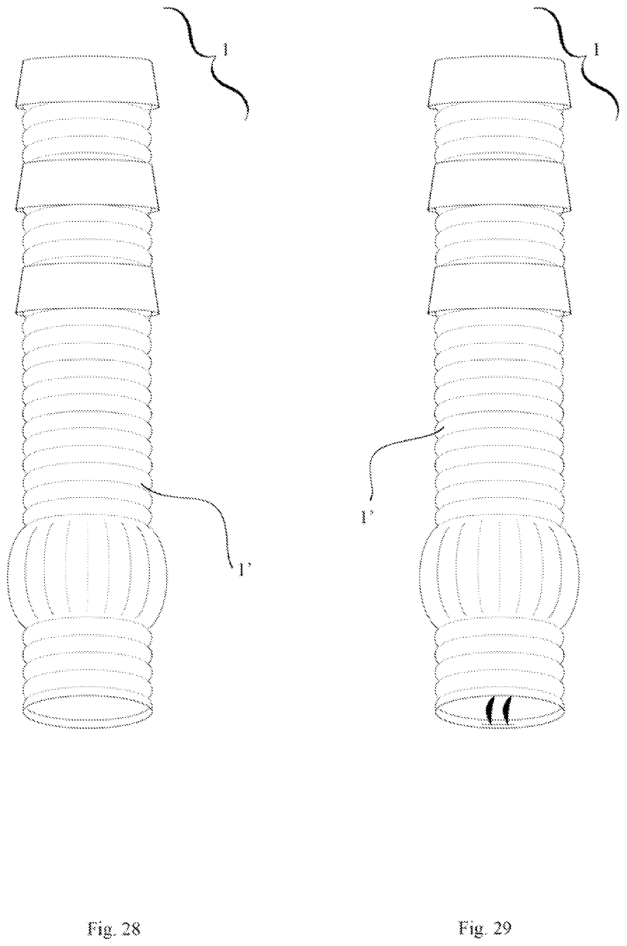

These embodiments correspond to the representations shown in the accompanying FIG. 14, FIG. 15, FIG. 16, FIG. 17, FIG. 18, FIG. 19, FIG. 20, FIG. 21, FIG. 22, FIG. 23, FIG. 24 and FIG. 25 and further provide the possibility that at least one reinforcement frame 2 is typically found at the point where said second portion is sutured to the distal aortic stump. If the ramifications 10 and a consecutive portion represented by an endoprosthesis are present, said reinforcement frame 2 is typically found at the transition point between the tubular portion having said branches 10 and the endoprosthesis-like portion. Alternatively to the reinforcement frame 2 present in said transition point, the prosthesis 1 may comprise a ring 11 for its adaptation to the descending thoracic aorta. In any case, the prosthesis 1 comprises at least a reinforcement frame 2 on said first prosthesis portion 1′.

Said embodiments also provide that, at the section shaped like a resilient tubular structure with said ramifications 10, a further ramification is present and in particular a branch 13 for perfusion of the prosthesis.

It is also of interest to note that such embodiments may include, as illustrated in FIG. 21, the presence of reinforcement frames also on at least one of said ramifications 10, for example if their stable adaptation to the stump of the brachiocephalic trunk, and/or of the left common carotid artery and/or of the left subclavian artery is necessary. The embodiments according to the present invention corresponding to a prosthesis as shown in the aforementioned figures are representative of a vascular prosthesis 1 particularly suitable for use in the treatment of aneurysms involving the aortic arch and/or the descending aorta.

A further embodiment according to the present invention provides that the prosthesis 1 comprises a segment shaped as a bifurcation 14 which is adapted to its implantation in the abdominal aorta site, at the aortic carrefour. Each branch of said bifurcation can have at least one reinforcement frame (2).

A further particular embodiment of the subject prosthesis is such as to make the latter particularly suitable for its implant at the height of the aortic root. In this case, said prosthesis will have at one end of said first tubular and resilient portion 1′, and in particular at the proximal end of the aortic implant, an integrated prosthetic valve and at the other end a reinforcement frame 2.

Yet another embodiment of the prosthesis 1 provides that the prosthesis 1 has an ampoule dilation along the tubular portion. This dilation mimics the shape of the aortic bulb and serves to facilitate the suture of the coronaries on such dilation of the prosthesis, avoiding the stretching of the coronary arteries.

At the other end of the prosthesis, at least one reinforcement frame 2 is present distal to the aortic bulb.

In all of its embodiments, all the portions comprised in the vascular prosthesis 1 are made of biocompatible materials. By way of non-limiting example, materials which can be used for making the subject prosthesis are biocompatible polymeric materials such as Dacron internally coated with collagen and PTFE. Engineered tissue with or without scaffolds as a support for the latter may also be used.

1. A vascular prosthesis (1) made of biocompatible material comprising at least one first portion (1′) that is tubular and resilient at least in longitudinal sense, wherein at least said tubular and resilient first portion (1′) has at least one reinforcement frame (2) shaped like a cuff-like covering, said reinforcement frame (2) being adapted to allow the execution of at least one suture line with separated stitches for the stable and durable anchoring of said prosthesis (1) to the vascular core (100) of interest, said reinforcement frame (2) defining, with the prosthesis portion from which the reinforcement frame departs, an interspace (7), the latter being delimited by the external surface of the prosthesis portion enclosed by said reinforcement frame (2) and by the internal surface of the latter, said reinforcement frame (2) having a first base and a second base, said first base being that closest, with respect to said second base, to the vascular core (100) end to which said frame (2) is to be sutured and to the tubular portion of the prosthesis 1′ with which the frame is in a continuity relationship.

2. The vascular prosthesis (1) according to claim 1, wherein the reinforcement frame (2) has a cylindrical profile, said reinforcement frame having a first base and a second base with diameter of equivalent extension, said diameter being greater than that of the tubular portion from which said reinforcement frame (2) departs at said first base.

3. The vascular prosthesis (1) according to claim 1 wherein the reinforcement frame (2) has frustoconical profile, said reinforcement frame (2) having a first base, with diameter analogous to that of the prosthetic portion from which said reinforcement frame (2) departs at said first base, and a second base, the latter having the edge free and a greater diameter than that of the first base, the latter being that closest, with respect to said second base, to the vascular core (100) end to which said frame (2) is to be sutured and to the tubular portion 1′ with which the frame is in a continuity relationship.

4. The vascular prosthesis (1) according to claim 1, wherein a reinforcement frame (2) is present on at least one end of the first portion (1′).

5. The vascular prosthesis (1) according to claim 4, wherein said reinforcement frame (2) has longitudinal cuts (9) and/or also a prosthetic extension (8) that departs without interruption from the first base of the reinforcement frame (2).

6. The vascular prosthesis (1) according to claim 1, comprising ramifications (10) suitable for the adaptation thereof at the arteries that originate from the aortic arch.

7. The vascular prosthesis (1) according to claim 1, wherein said prosthesis comprises a second portion (1″) of prosthesis (1), said second portion (1″) of prosthesis (1) being represented by an endoprosthesis shaped like a self-expandable stent that departs from said first portion (1′) without interruption.

8. The vascular prosthesis (1) according to claim 1, wherein the first portion (1′) of prosthesis (1) comprises radio markers (12).

9. The vascular prosthesis (1) according to claim 1, wherein at least two reinforcement frames (2) are present, one on each end of said first portion (1′) of prosthesis (1), said reinforcement frames being oriented mirrored with respect to each other.

10. The vascular prosthesis (1) according to claim 6, wherein the first portion (1′) of prosthesis (1) comprises radio markers (12), and wherein a reinforcement frame is present at the transition point between the portion section (1′) comprising the ramifications (10) and that comprising the radio markers (12).

11. The vascular prosthesis (1) according to claim 6, wherein said prosthesis comprises a second portion (1″) of prosthesis (1), said second portion (1″) of prosthesis (1) being represented by an endoprosthesis shaped like a self-expandable stent that departs from said first portion (1′) without interruption, and wherein a reinforcement frame (2) is present at the transition point between the first portion (1′) comprising the ramifications (10) and the second portion (1″) shaped like a self-expandable stent.

12. The vascular prosthesis (1) according to claim 6, wherein said prosthesis comprises a second portion (1″) of prosthesis (1), said second portion (1″) of prosthesis (1) being represented by an endoprosthesis shaped like a self-expandable stent that departs from said first portion (1′) without interruption, and wherein a ring (11) is present at the transition point between the first portion (1′) comprising the ramifications (10) and the second portion (1″) shaped like a self-expandable stent.

13. The vascular prosthesis (1) according to claim 6, wherein, in the first prosthesis portion (1′), at least one branch (13) is present for the perfusion of the prosthesis.

14. The vascular prosthesis (1) according to claim 6, wherein at least one reinforcement frame (2) is present on at least one of the ramifications (10).

15. The vascular prosthesis (1) according to claim 1, comprising a structure shaped like a bifurcation (14), the latter having, on each branch, at least one reinforcement frame (2).

16. The vascular prosthesis (1) according to claim 1 wherein a prosthetic valve can be situated at one end of the first portion (1′) of prosthesis (1).

17. The vascular prosthesis (1) according to claim 16 wherein on the body of the first portion (1′) of prosthesis (1), an expansion is present, shaped like a vial, adapted to mimic the anatomy of the aortic sinuses of the aortic root.

18. The vascular prosthesis (1) according to claim 1 wherein said prosthesis comprises a plurality of suture wires (3) with double needle (4), integrated with at least one reinforcement frame (2), each of said suture wires (3) defining an overturned U.

19. The vascular prosthesis (1) according to claim 1 for use in a method for treating arterial pathologies.

20. The vascular prosthesis (1) according to claim 19 for use in a method for treating aortic aneurism or aortic dissection.
